(19) United States
(12) Patent Application Publication
(10) Pub. No.: US 2026/0226012 A1
(43) Pub. Date: Aug. 6, 2026

(54) PROCESS FOR TREATING IMINIUM SALT-BASED IONIC LIQUIDS

(71) Applicant: Technikum Laubholz GmbH, Göppingen (DE)

(72) Inventor: Willi KANTLEHNER, Aaeln (DE)

(21) Appl. No.: 19/149,645

(22) PCT Filed: Jan. 19, 2024
(86) PCT No.: PCT/EP2024/051243

§ 371 Date: Jul. 21, 2025

(30) Foreign Application Priority Data
Jan. 20, 2023 (DE) 10 2023 101 455.2

(51) Int. Cl. C07D 233/02; C07C 53/126

(52) U.S. Cl. CPC C07D 233/02; C07C 53/126

(57) ABSTRACT

The present invention relates to a process for treating an ionic liquid in which process an iminium salt, particularly in the form of an imidazolium salt, is reacted with an alcoholate to give an iminium alcoholate adduct, the salt formed from the cation of the alcoholate and the anion of the iminium salt is then separated off, and an iminium salt is then regenerated by addition of an acid. Owing to the much lower polarity compared to the iminium salt, the iminium alcoholate adduct allows easy separation of polar impurities or a clean exchange of the anion of the iminium salt, if the added acid has a different anion from that originally present in the iminium salt.

The present invention relates to a process for treating an ionic liquid in which process an iminium salt, preferably in the form of an imidazolium salt, is reacted with an alcoholate to give an iminium alcoholate adduct, the salt formed from the cation of the alcoholate and the anion of the iminium salt is then separated off, and an iminium salt is then regenerated by addition of an acid. Owing to the much lower polarity compared to the iminium salt, the iminium alcoholate adduct allows easy separation of polar impurities or a clean exchange of the anion of the iminium salt (=anion metathesis) if the added acid has a different anion from that originally present in the iminium salt.

### PRIOR ART

Iminium salt-based ionic liquids are used as solvents or reaction media in many processes (see, e.g., Ionic Liquids for Clean Technology, K. R. Seddon, J. Chem. Tech. Biotechnol. 1997, 68, 351-356; Room Temperature Molten Salts, N. N. Ene, Roum. Chem. Q. Rev. 1993, 1, 333-351). After the desired products are separated off, these applications result in ionic liquids that are contaminated by unreacted starting materials, added additives, or reaction products.

Contaminated ionic liquids are difficult to recycle due to their special properties. The compounds usually have boiling points or sublimation points that are well above 300° C. Only ionic liquids containing special anions such as ⁻N(SO₂F)₂ are reasonably stable at these temperatures, so that they can often be distilled on a small scale. However, this is also associated with considerable energy consumption.

Given the above, there is a need for a generally applicable cleaning or recycling process that can also be carried out at lower temperatures (<200° C.).

A further problem with the use of ionic liquids is that iminium salt-based ionic liquids available on a technical scale mostly contain chloride ions as an anionic component, wherein 1-butyl-3-methylimidazolium chloride (1), which is produced on a large scale by BASF SE, can be cited as an example.

However, many processes require iminium salt-based ionic liquids that contain other, e.g., reactive, highly nucleophilic anions such as iodide, thiocyanate, or cyanide. Such reactive ionic liquids can, in principle, be produced from chloride salts of iminium salt-based ionic liquids via reactions known as “anion metathesis”.

For guanidinium salt-based ionic liquids, a large number of anion exchange methods have been developed, which are briefly presented below:

### a) Double Reaction

A solution of salt AX, whose anions X⁻ are to be exchanged, is added with a solution of another salt BY, which contains the desired anion Y⁻. The cation B⁺ of the salt BY is selected such that the salt BX is insoluble and precipitates. After the separation of the salt BX, the resulting solution of the desired ionic liquid AY is evaporated, and AY is isolated as a residue. A large number of guanidinium salts with different anions were prepared according to this principle (cf., e.g., W. Kantlehner, J. Mezger, I. Tiritiris, R. Kreß, W. Frey, Z. Naturforsch. 2021, 76b, 133-162).

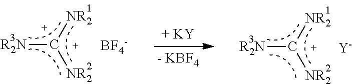

Guanidinium salts with anions containing organofluorine groups are poorly soluble in water. If aqueous solutions containing alkali salts with organofluorine anions, such as CF₃SO₃Li, CF₃SO₃Na, C₄H₉SO₃Na, or N(CF₃SO₂N)₂Na, are added to water-soluble guanidinium salts, the guanidinium salts precipitate (see EP 1363345 A2 or DE 10325051A1).

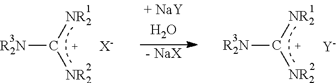

### b) Decomposition of Acid-Labile Anions

In the case of salts containing acid-sensitive anions, such as carbonates or hydrogen carbonates, the anion can be exchanged by adding an acid, leading to the formation of carbonic acid, wherein, by subsequent elimination of carbon dioxide, the ionic liquid is formed with the anion of the added acid.

c) Complexation of Anions with Lewis Acids

This reaction generates guanidinium tetrafluoroborates from corresponding guanidinium fluorides and guanidinium chlorides with boron trifluoride etherate (cf., e.g., W. Kantlehner, J. Mezger, I. Tiritiris, R. Kreß, W. Frey, Z Naturforsch. 2021, 76b, 133-162).

### d) Alkylation of the Anions of Ionic Liquids

In the case of salts with strongly nucleophilic anions, anion exchange can be achieved by alkylating the anion. With less nucleophilic anions, alkylation can still be achieved with very strong alkylating agents. Thus, guanidinium chlorides, e.g., are converted into guanidinium tetrafluoroborates with triethyloxonium tetrafluoroborate with the release of volatile ethyl chloride and diethyl ether (see e.g., W. Kantlehner, J. Mezger, I. Tiritiris, R. Kreß, W. Frey, Z Naturforsch. 2021, 76b, 133-162).

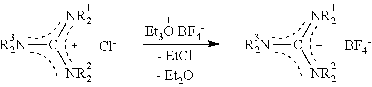

In addition, for the anion metathesis of ionic liquids based on imidazole salts in particular, other possibilities exist as well, such as the reaction with silver salts (with precipitation of silver halides), the reaction with HPF₆ to form hexafluorophosphate salts, or the reaction with acetate-loaded ion exchange resins to form acetate salts.

However, as this compilation shows, there is no generally applicable anion metathesis method for iminium salt-based ionic liquids that allows the introduction of any anion, regardless of the type of anion to be exchanged.

The difficulty with the methods described above also lies in the fact that for the reaction, there must be differences in the specific properties of the ionic liquid used as the starting material and the ionic liquid obtained as the product, suitable reagents such as alkylating agents must be available, or complexable or decomposable anions must be present in the ionic liquid used as the starting material.

Thus, there is also a need for a process that is as generally applicable as possible, with which ionic liquids with more easily accessible anions, such as halide and in particular chloride, can be converted into corresponding ionic liquids with other anions in a simple and cost-effective manner.

The present invention addresses this need.

Finally, reference should be made to the publication of Bredereck H. et al, Chem. Ber. 1965, 98, p. 1078ff., in which the reaction of N,N,N′,N′-tetramethylformamidinium methyl sulfate with sodium methylate to form the aminal ester bis-dimethylaminomethoxymethane is described. Subsequently, in Angew. Chem Int. Ed. Engl. 1967, p. 311, analogous aminal esters were obtained by reaction with ethylates, i-propylates, and i-butylates. Korshin et al describe in Zh. Org. Khim. 1993, 29, p. 577-87, the reaction of 4-anilino-1,3-diphenyl-imidazolium chloride with sodium methanolate to give the corresponding aminal ester.

### DESCRIPTION OF THE INVENTION

In the investigations on which the present invention is based, it was surprisingly found that not only guanidinium salt-based ionic liquids, but also iminium salt-based ionic liquids in general, can be converted to iminium alcoholate adducts belonging to the orthoamide class of compounds by the addition of alcoholates. Owing to their electrically neutral structure, these adducts can be purified from polar impurities and salts. In addition, such a treatment has the advantage that an acid can be added at the orthoamide or adduct stage, which has a different anion than the ionic liquid from which the orthoamide or adduct was produced, which corresponds to an anion exchange (anion metathesis) in the overall conversion.

Accordingly, the present invention relates to a process for the treatment of an ionic liquid based on a substituted or unsubstituted iminium salt (Im⁺X⁻), wherein the iminium salt is reacted in a first step i) with an alcoholate to form an electrically neutral iminium alcoholate adduct, the salt formed from the cation of the alcoholate and the anion X⁻ of the iminium salt is separated off in a second step ii), and the electrically neutral iminium alcoholate adduct is reacted, in a third step iii), with an acid HY forming an iminium salt Im⁺Y⁻, wherein X and Y may be identical or different. This process is further described below via a reaction scheme, wherein the iminium salt is in the form of an imidazolium salt (Im⁺X⁻, 1), which represents a preferred iminium salt in the context of the present invention. In the reaction equation below, compound 2 is a non-charged or electrically neutral imidazole alcoholate adduct and compound 3 is the imidazolium salt Im⁺Y⁻.

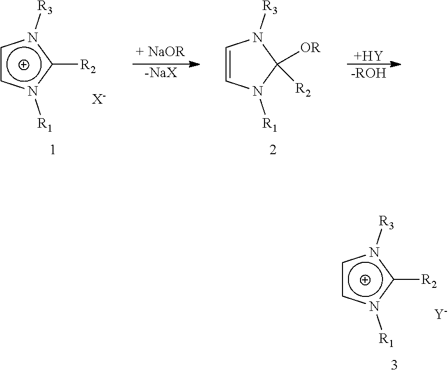

As mentioned before, the iminium salt, which is subjected to the treatment according to the invention, is present in a preferred embodiment as an imidazolium salt. However, the iminium salt may also have a cation that is not based on an imidazole ring, such as an N,O-bisalkylated pyrrolidone, R¹—C(NR²₂)₂⁺, R¹—C(NR²)(SR₂)⁺, or a guanidinium cation (C(NR²₂)₃⁺), wherein R² may be the same or different in the structures shown and denotes a hydrocarbon radical, preferably in the form of an alkyl radical, and wherein R¹ denotes a hydrocarbon radical, preferably in the form of an alkyl radical. Other possible iminium salts that can be treated in the process described herein include the 1,3-dialkyl-1,3,4-triazolium salts (4), 2,3,5-trialkyl-1,3-oxazolium salts (5), 2,3,5-trialkyl-1,3,4-oxdiazolium salts (6), 2,3,5-trialkyl-1,3-thiazolium salts (7), 2,3,4-trialkyl-1,3,4-thiadiazolium salts (7), 1,2-dialkyl-pyrimidinium salts (9), and 1,2,3-trialkyl-3,4,5,6-tetrahydro-pyrimidinium salts (10) shown below:

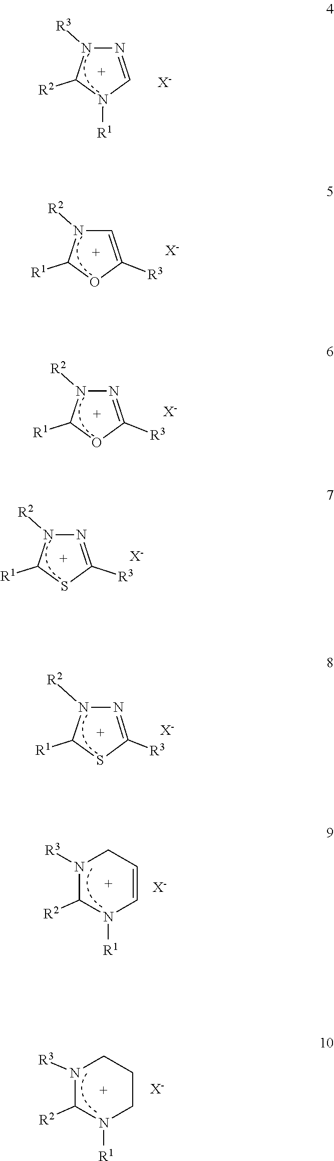

In a first embodiment, X and Y are the same (see the following figure using the example of an ionic liquid with an imidazolium cation), i.e., compound 1 is the same as compound 3a. Such a process is suitably designed as a process for purifying the reacted ionic liquid 1, in which polar, and in particular ionic impurities, are separated from the iminium alcoholate adduct in step ii) with the salt. Impurities to be separated include impurities that occur in typical applications of ionic liquids, e.g., formate, acetate, butanoate, chloride, bromide, iodide, or tetrafluoroborate.

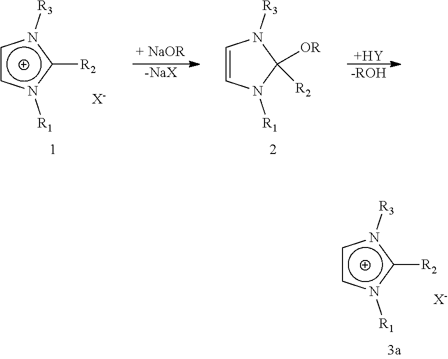

The possibility of separating off impurities and of purifying is exemplified by the properties of N,N,N′,N′,N″,N′″-hexamethylguanidinium chloride 1 and the tris(dimethylamino)ethoxymethane 2 derived therefrom:

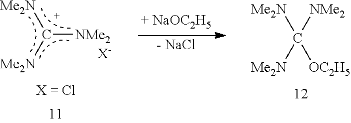

The guanidinium salt 11 has a melting point of 296° C. (see W. Kantlehner, E. Haug, W. W. Mergen, P. Speh, T. Maier, J. J. Kapasakalidis, H.-J. Bräuner, H. Hagen, Synthesis 1983, 13, 904-905) and is insoluble in diethyl ether, ethyl acetate, benzene, or cyclohexane. When sodium ethylate is added, the orthoamide derivative 5 is formed from the guanidinium salt 11 as an adduct in form of a colorless to pale yellow liquid with a boiling point of 73° C./10 Torr (see W. Kantlehner, W. W. Mergen, Synthesis 1979, 11, 343-344), which is miscible with ether, ethyl acetate, benzene, and cyclohexane in any ratio.

In a second embodiment, X and Y are different, i.e., compound 1 is different from compound 3 as far as the anion is concerned. Such a process is designed as a process for producing an ionic liquid Im⁺Y⁻ from an ionic liquid Im⁺X⁻, in which the anion Y⁻ is different from the anion X⁻, which is contained in the starting product. In this embodiment, it is preferred if X⁻ is a halide, in particular in the form of chloride, or the anion of a C1 or C2 carboxylic acid (i.e., formate or acetate). It is particularly preferred that X⁻ is a chloride.

If an alcoholic alcoholate solution is used as the “alcoholate source” in step i), it may be suitable if excess alcohol is removed following the reaction to avoid subsequently solvent mixtures with the alcohol, which may promote less complete separation of the impurities or salts. The alcohol can be separated here from the mixture by any process suitable for removing it, wherein distillation or vacuum treatment may be named by way of example. Alternatively, an alcohol can be used as a solvent for the ionic liquid (preferably in the form of methanol or ethanol), for example, and to facilitate the separation of the salt in step ii) via filtration.

The alcoholate which is added to the substituted or unsubstituted imidazolium salt in step i) according to the process of the invention is suitably a C1-C4 alcoholate, wherein methanolate and ethanolate are particularly preferred. An alkali metal cation, more preferably in the form of sodium or potassium, can be used as the cation of the alcoholate. Sodium is usually preferred here for reasons of cost and availability.

The processes according to the first or second embodiment may suitably be further designed such that an aprotic, non-polar solvent is added to the mixture in step ii) before the salt formed from the cation of the alcoholate and the anion X⁻ of the iminium salt is separated off. With respect to the type of aprotic, non-polar solvent, the present invention is not subject to any relevant limitations, i.e., aliphatic or aromatic hydrocarbons may be used as solvents, wherein “aliphatic” includes “cycloaliphatic” forms. Suitable aliphatic or aromatic hydrocarbon solvents are, for example, hexane, pentane, cyclohexane, cyclopentane, toluene, benzene, or xylene, or mixtures of the solvents mentioned. Other suitable solvents are ethers such as diethyl ether or methyl t-butyl ether, or esters such as ethyl acetate or ethyl propionate, and nitriles such as acetonitrile. Halogenated and particularly chlorinated hydrocarbons such as dichloromethane or chloroform are also suitable, but less favorable in view of toxicological considerations. However, the solvent is not limited to the specific examples mentioned, but rather it is of relevance that the iminium alcoholate adduct dissolves in the solvent, while polar impurities are not dissolved and accumulate as a residue.

Furthermore, it is desirable, but not essential, that the solvent has a lower boiling temperature than the iminium alcoholate adduct, since in this case, the solvent can subsequently be removed from the mixture by distillation. “Distillative” here also includes cases of treatment under reduced pressure, in which the solvent is vaporized and then condensed again by appropriate cooling.

Alternatively, it is possible, in particular if a carboxylic acid such as a fatty acid is used as HY, that the process is carried out in the presence of water as solvent, wherein a protic solvent such as alcohol (e.g., as a solvent for an alcoholate used) is additionally present. The water may form the main part of the solvent and may, for example, make up more than 80% by weight, preferably more than 90% by weight, and even more preferably more than 93% by weight of the total amount of solvent in the mixture.

The separation of the salt formed from the cation of the alcoholate and the anion of the iminium salt may be carried out in step ii) by means of any process suitable for the separation of solid or denser residues. In one example, the separation of the salt is carried out by filtration, preferably at ambient temperature (20 to 28° C.) or a lower temperature above the deposition temperature of the iminium alcoholate adduct. The “deposition temperature” is the temperature at which the iminium alcoholate adduct is no longer dissolved, that is, as a result, e.g., of crystallization or phase separation. In another embodiment, the separation is carried out by decanting the solution of the iminium alcoholate adduct in an aprotic, non-polar solvent, optionally after centrifugation to concentrate undissolved components at the bottom of a reaction vessel.

For steps i) and ii), the temperature also has a certain significance, which on the one hand should be selected such that the iminium salt and the iminium alcoholate adduct produced therefrom are present in liquid form, and on the other hand the temperature should not be so high that the iminium alcoholate adduct is subject to relevant decomposition. It is preferable here if the temperature for steps i) and ii) is kept below 120° C., in particular below 100° C., and further preferably below 80° C.

As already mentioned, the anion of the iminium salt used in step i) is preferably a halide and particularly preferably chloride if a product with a Y⁻ different from X⁻ is produced in the process according to the invention. But even in a process in which X⁻ and Y⁻ are the same, X⁻ can be present as halide or chloride. In this case, however, a significantly higher range of anions for X⁻ is conceivable, as will be described below. In particular, the anion can also be a carboxylate, alkyl sulfate, alkyl sulfonate, or aryl sulfonate.

In the processes according to both embodiments described above, the acid added in step iii) may be any acid which reacts with the imidazole alcoholate orthoamide under release of alcohol (ROH), i.e., it must be a protic acid. Suitable acids of this type include, but are not limited to, hydrochloric acid, hydrobromic acid, hydroiodic acid, C1-C18 carboxylic acids, i.e., e.g. formic acid, acetic acid, or propionic acid, methanesulfonic acid, dialkylphosphonic acid, in particular in the form of dimethyl- or diethylphosphonic acid, thiocyanuric acid, hydrogen cyanide, cyanoic acid, nitric acid, nitrous acid, sulfuric acid, alkylsulfuric acid, alkyl- or arylsulfonic acid, HN(SO₂CF₃)₂, trifluoroacetic acid, and trifluoromethanesulfonic acid.

Y⁻ in the compound 3 specified above may also be an adduct of a carboxylic acid and a deprotonated carboxylic acid (in the form of R—CO⁻HOCOR), in which the proton of the carboxylic acid is stabilized by the carboxylate group of the deprotonated carboxylic acid.

The acid added in step iii) can be added at a suitable temperature, which can be adjusted depending on the reactivity of the iminium alcoholate adduct and the stability against undesired decomposition. In one embodiment, the acid is added at ambient temperature (20 to 28° C.). In another embodiment, the acid is added at a reduced temperature, e.g. at −30° C. to 15° C. or −20° C. to 0° C. Subsequently, the reaction mixture can be heated to a higher temperature, e.g., 45 to 100° C. or 60 to 90° C., to complete the reaction.

If the iminium salt used in the process according to the invention is an imidazolium salt, it is preferred if the imidazolium cation contained in the salt is substituted at the nitrogen atoms (1′- and 3′-position) and preferably unsubstituted at the 4′- and 5′-position. The carbon atom at the 2′ position may be unsubstituted (i.e., have an additionally bonded hydrogen atom, R²═H) or substituted, for example with an alkyl group which may contain one or more substituents selected from alkyl, in particular C1-C6 alkyl, alkoxy, in particular C1-C6 alkoxy, aryl, in particular phenyl. Alternatively, the carbon atom at the 2′ position may be substituted with an aryl radical (e.g., in the form of phenyl) or with NR₂, wherein R may be an alkyl or aryl radical, and the two Rs may be identical or different. Preferably, R is a C1-C4 alkyl radical, and most preferably R=methyl.

In a particularly preferred embodiment, the imidazolium cation of the imidazolium salt is substituted in the 1′- and 3′-position (corresponding to the radicals R¹ and R³ in the reaction scheme above) or in the 1′-, 2′- and 3′-position (corresponding to the radicals R¹, R², and R³ in the reaction scheme above) with (C1-C6)-alkyl groups, wherein a 2′-position can also be substituted with aryl or alkylaryl in addition to alkyl, and the alkyl group can have an alkyloxy or aryloxy substituent. R¹ and R³ may be the same or different. In still another preferred embodiment, the imidazolium cation of the imidazolium salt is selected from the group consisting of 1-ethyl-3-methylimidazolium, 1,3-dimethylimidazolium, 1-butyl-3-methylimidazolium, 1-butyl-3-ethylmethylimidazolium, 1,3-diethylimidazolium, 1-ethyl-3-isopropylimidazolium, 1-allyl-3-methylimidazolium, 1-allyl-3-ethylimidazolium, 1-allyl-3-isopropylimidazolium, and 1-allyl-3-butylimidazolium cations.

The anion X⁻ of the imidazolium salt included in the process according to the invention is preferably selected from halides, in particular chloride, bromide or iodide, the carboxylate of a C1-C18 carboxylic acid, in particular a C1-C12 carboxylic acid such as acetate, formate, propionate, butanoate, or octanoate, a dialkyl phosphate, in particular in the form of dimethyl or diethyl phosphate, dialkyl phosphonate such as dimethyl or diethyl phosphonate methylsulfonate, thiocyanate, cyanate, nitrate, nitrite, ⁻N(SO₂CF₃)₂, ⁻N(SO₂C₄F₉)₂, and trifluoromethanesulfonate.

In step iii) of the process described, the alcohol is regenerated from the added acid and the alcoholate bound in the iminium alcoholate adduct, which is usually not desirable for further use of the imidazolium salt formed as an ionic liquid. It is therefore preferred if the process according to the invention additionally comprises a step in which the alcohol is removed from the product formed. For suitable processes for this purpose, reference can be made to the above specifications on processes for removing excess alcohol, wherein distillation and/or vacuum treatment can be indicated as particularly suitable.

If the iminium cation of the treated ionic liquid has an imidazolium cation which has an alkyl substituent in the 2′ position with a hydrogen substituent bonded in the α-position of the C atom directly bonded to the 2-imidazole carbon atom (13), a 2-alkylidene imidazole 15 can be formed in equilibrium with the imidazole-alcoholate adduct 14 by elimination of alcohol:

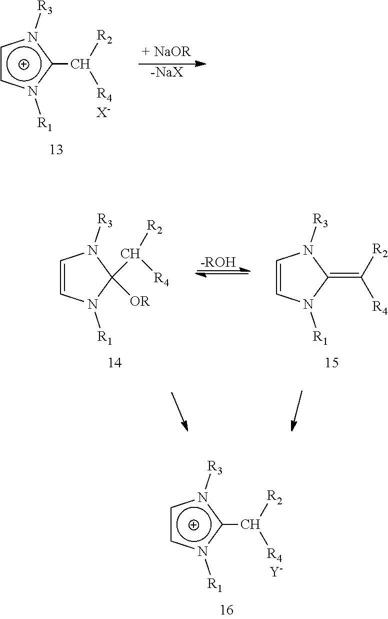

In formula 13, R¹ and R³, which may be identical or different, have the same meaning as described above, and R² and R⁴, which may be identical or different, have the same meaning as R² above, including the possibility that R² and R⁴═H. It is known that such 2-alkylidene imidazoles often do not exhibit high thermal stability, so that isolation of these products is usually only possible with significant yield losses (see, e.g., WO 2007/131498 A1). It is therefore preferred if imidazole alcoholate adducts from which alcohol can be cleaved are treated under very mild thermal conditions, e.g., of less than 120° C. and in particular less than 100° C. in steps i) and ii). In this way, a significant decomposition via the 2-alkylidene imidazoles is avoided. For this purpose, it is further preferred if the processing is carried out under conditions in which the 2-alkylidene imidazole formed constitutes a proportion of the total amount of imidazole alcoholate adduct and 2-alkylidene imidazole of 2 to 80 wt. % and more preferably 5 to 30 wt. %. In particular, it is not intended to isolate pure 2-alkylidene imidazole in the context of the process described here, which is not necessary anyway, since both the imidazole-alcoholate adduct and the 2-alkylidene imidazole are converted into an imidazolium salt by the addition of acid.

In such a case, in order to be able to subsequently properly adjust the required amount of acid to be added and not to introduce excess acid into the system, it may be useful if the process additionally comprises a step of determining the amount of acid to be added in step iii) which is required to completely convert the mixture of imidazole alcoholate adduct of 2-alkylidene-imidazole into an imidazolium salt (i.e., a stoichiometric amount based on the molar amount of imidazole units in the mixture). Such an adjustment is possible, for example, by determining the ratio of the imidazole alcoholate adduct to 2-alkylidene imidazole using ¹H NMR or by titration.

In a further aspect, the present invention relates to an imidazolium salt (Im⁺Y⁻), obtainable with the process described above, and wherein Y⁻ is selected from the group comprising bromide, iodide, a C3-C18 carboxylic acid, a dialkyl phosphonate, particularly in the form of dimethyl or diethyl phosphonate, a dialkyl phosphate, particularly in the form of dimethyl or diethyl phosphate, methylsulfonate, thiocyanate, cyanate, nitrate, nitrite, an alkyl sulfate, an alkyl or aryl sulfonate, particularly in the form of the dimethyl or diethyl derivatives, ⁻N(SO₂CF₃)₂ and trifluoromethanesulfonate. If Y⁻ is a C3-C18 carboxylic acid (and particularly a C10 to C18 carboxylic acid), it is also possible that Y⁻ is present in the form of an adduct of deprotonated carboxylic acid and carboxylic acid, as indicated above. Such an imidazolium salt is characterized by a high purity of the anion, for example at least 90 mol % (based on the total amount of anions in the imidazolium salt), more preferably at least 95 mol %, still more preferably at least 98 mol % and still more preferably at least 99 mol % of the anion indicated (e.g. thiocyanate).

In individual cases, it is possible, in particular if water is used as the solvent for the reaction of the ionic liquid with the alcoholate, and an anion Y⁻ having a large non-polar portion (such as a carboxylate, particularly of a fatty acid, having at least 4 C atoms, preferably at least 8 C atoms and more preferably 10 to 18 C atoms) is used, to perform the process such that the separation of the salt formed from the cation of the alcoholate and the anion X⁻ of the iminium salt according to step ii) is carried out after the addition of an acid HY to form an iminium salt Im⁺Y⁻ according to step iii). In this case, the desired product salt of the ionic liquid can be obtained after precipitation and/or crystallization from the reaction mixture and separation of the dissolved salts formed from the alcoholate and the anion X−.

With the process described, it is possible to regenerate ionic liquids in a simple manner, wherein polar impurities or salts that cannot be removed from the ionic liquids by extraction with non-polar solvents are separated. In addition, the process described allows the simple and cost-effective production of iminium salts with uncommon and commercially unavailable anions, which would otherwise have to be produced using significantly more complex processes.

In the following, the present invention is illustrated in more detail by means of some exemplary embodiments, which, however, should not be considered as limiting or restricting the scope of protection of the application in any way.

### EXAMPLES

### Example 1: Preparation of 1-butyl-3-methylimidazolium palmitate-palmitic acid adduct

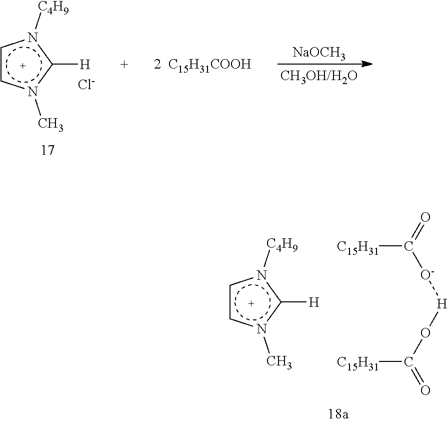

To a suspension of 1.76 g (0.01 mol) of 1-butyl-3-methylimidazolium chloride (17) and 2.56 g (0.01 mol) of palmitic acid in 30 mL of water, 1.8 g of a 30% solution of sodium methylate (0.01 mol) in methanol is added dropwise while stirring. A colorless precipitate forms. The mixture is heated to boiling while stirring to form an almost clear solution, which is filtered while hot. The waxy, colorless mass that precipitates from the filtrate during cooling is filtered off by suction and washed twice with 20 mL of methanol each time and dried. Yield: 2.11 g (64.7%, based on the palmitic acid added) 18a with mp 54° C. A small amount (approx. 0.15 g) of a colorless product precipitates from the methanol during cooling and is filtered off by suction. Evaporation of the filtrate yields a further 1.1 g (33.7%, based on the palmitic acid added) of slightly impure 18a with mp 48° C.

C₄₀H₇₈N₂O₄ (651.04) Calcd. C, 73.79; H, 12.08; N, 4.30; found C, 74.24; H, 11.95; N, 4.39.

### Example 2: Preparation of 1-ethyl-3-methylimidazolium octanoate (18b)

### Preparation of 18b with Diethyl Ether and Methanol as a so/Vent Mixture

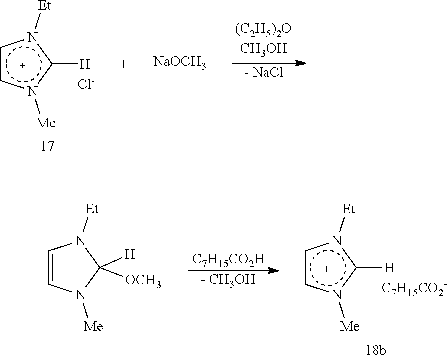

To 2.92 g (0.02 mol) of EMIMCI (17) in 20 mL of methanol and 10 mL of diethyl ether, 3.60 g (0.2 mol) of sodium methylate solution (30% in methanol), which was previously diluted with 10 mL of methanol, is added while stirring. The colorless precipitate formed is separated after 15 min. The filtrate is mixed with 2.88 g (0.02 mol) of caprylic acid (octanoic acid) in 10 mL of ether while stirring. After standing for 2 hours, the clear mixture is evaporated in vacuo, leaving a gelatinous, glassy mass which is freed from the solvents. The residue is boiled with 30 mL of acetonitrile for 15 min while stirring, wherein the gelatinous mass is disaggregated from time to time. After cooling, this is filtered off by suction. The filter residue is washed with 15 mL of ether. The filtrate (acetonitrile/ether) is filtered through a pleated filter and then evaporated in vacuo. Yield: 3.4 g (73%) of EMIMOCT (18b), yellow oil with n²⁰_{D}=1.4806 (commercial product Fa. proionic >98%, n²⁰_{D}=1.4805).

### Preparation of 18b with Acetonitrile and Methanol as a Solvent Mixture

2.92 g (0.02 mol) of 1-ethyl-3-methylimidazolium chloride (17) in a mixture of 10 mL of methanol and 10 mL of acetonitrile are added dropwise while stirring with 3.60 g (0.02 mol) of a 30% sodium methanolate solution, which was made up to 10 mL before the addition. Immediately, a colorless precipitate forms. After the addition is complete, stirring is continued for a further 5 minutes, and then a filtration is performed.

The filtrate is added with 2.88 g (0.02 mol) of octanoic acid in 5 mL of acetonitrile under stirring, wherein a voluminous, gelatinous precipitate is formed under heating. After stirring for approx. 20 minutes, a filtration with suction is carried out. The filtrate is evaporated in vacuo to obtain a voluminous gelatinous mass, which is heated with 15 mL of acetonitrile for 15 min under reflux with stirring, wherein the gelatinous, lumpy mass is disaggregated from time to time. After cooling, this is filtered off by suction.

A small amount of gelatinous mass precipitates from the filtrate and is filtered off. After evaporation of the filtrate in vacuo, 2.64 g (52%) of 1-ethyl-3-methylimidazolium octanoate (18b) remain as a pale yellow oil, n²⁰_{D}=1.4801 (commercial product Fa. proionic >98%, n²⁰_{D}=1.4805).

### Production of 18b in Methanol

2.92 g (0.02 mol) of 1-ethyl-3-methylimidazolium chloride (17) (EMIMCI) in 10 mL of methanol is added with 3.60 g (0.02 mol) of sodium methanolate solution (30% in methanol), which has been made up to 10 mL with methanol, while stirring. Immediately, a colorless precipitate (NaCl) forms, which is filtered off after 15 minutes of stirring.

The filtrate is mixed with 2.88 g (0.02 mol) of octanoic acid in 5 mL of methanol while stirring. The mixture remains clear and is evaporated, towards the end in vacuo. The residue is heated with 15 mL of acetonitrile under reflux while stirring. On cooling, a colorless, waxy solid precipitates, which is filtered off by suction and washed with ether. After concentration of the filtrate—towards the end in vacuo—an oily substance remains alongside a glass-like jelly. The mixture is heated with 10 mL of acetonitrile while stirring to produce a clear solution. As it cools, a small amount of glassy mass precipitates and is filtered off by suction. Evaporation of the filtrate in vacuo yields 2.28 g (45%) of 1-ethyl-3-methylimidazolium octanoate 18b as a pale yellow oil, with n²⁰_{D}=1.4803 (commercial product Fa. proionic >98%, n²⁰_{D}=1.4805).

1.-15. (canceled)

16. An imidazolium salt Im+Y−, wherein Y− is an adduct of a deprotonated C3-C18-carboxylic acid and a corresponding C3-C18-carboxylic acid.

17. The imidazolium salt according to claim 16, wherein Y− is an adduct of a deprotonated C10-C18-carboxylic acid and a corresponding C10-C18-carboxylic acid.

18. The imidazolium salt according to claim 16, wherein the imidazolium salt is substituted with (C1-C6)-alkyl groups in the 1′- and 3′-position or in the 1′-, 2′- and 3′-position, wherein the 2′-position may, in addition to alkyl, also be substituted with aryl or alkylaryl, and the alkyl group may have an alkyloxy or aryloxy substituent.

19. The imidazolium salt according to claim 16, wherein the imidazolium cation is selected from the group consisting of 1-ethyl-3-methylimidazolium, 1,3-dimethylimidazolium, 1-butyl-3-methylimidazolium, 1-butyl-3-ethylmethylimidazolium, 1,3-diethylimidazolium, 1-ethyl-3-isopropylimidazolium, 1-allyl-3-methylimidazolium, 1-allyl-3-ethylimidazolium, 1-allyl-3-isopropylimidazolium, 1-allyl-3-butylimidazolium cations.

20. A method for the preparation of an ionic liquid according to claim 16, characterized in that an imidazolium salt (Im+X−) is reacted in a first step i) with an alcoholate to form an electrically neutral imidazolium alcoholate adduct, the salt formed from the cation of the alcoholate and the anion X− of the imidazolium salt is separated off in a second step ii), and the electrically neutral imidazolium alcoholate adduct is reacted with an acid HY to form an imidazolium salt Im+Y− in a third step iii), wherein X and Y are different, and wherein Y− is the adduct of a carboxylic acid and a deprotonated carboxylic acid.

21. The method according to claim 20, characterized in that, before the salt formed from the cation of the alcoholate and the anion X− of the imidazolium salt is separated off, an aprotic, non-polar solvent, preferably in the form of an aliphatic or aromatic hydrocarbon, a halogenated hydrocarbon, an ether, or an ester, is added to the mixture in step ii).

22. The method according to claim 20, characterized in that the separation of the salt is carried out by filtration.

23. The method according to claim 20, characterized in that the anion of the imidazolium salt used in step i) is a carboxylate, alkyl sulfate, alkyl or aryl sulfonate, tetrafluoroborate, dialkyl phosphonate, or a halide.

24. The method according to claim 20, characterized in that a C3-C18-carboxylic acid is used as the acid in step iii).

25. The method according to claim 20, characterized in that in step i), the alcoholate is a C1-C4 alcoholate.

26. The method according to claim 20, further comprising a measure for removing the alcohol formed from the acid and the imidazolium alcoholate adduct in step iii).

27. The method according to claim 20, wherein water is used as a solvent for the reaction of the ionic liquid with the alcoholate, and a carboxylate of a fatty acid having at least 4 C atoms is used as the anion Y−, and wherein the process is carried out in such a way that the separation of the salt formed from the cation of the alcoholate and the anion X− of the imidazolium salt according to step ii) takes place after the addition of the fatty acid HY having at least 4 C atoms to form an imidazolium salt Im+Y− according to step iii).

28. The method according to claim 22, characterized in that the filtration is performed at ambient temperature (23° C.) or a lower temperature above the precipitation temperature of the imidazolium alcoholate adduct.

29. The method according to claim 23, characterized in that the anion is in the form of chloride.

30. The method according to claim 20, characterized in that the alcoholate is a methanolate or ethanolate.

31. The method according to claim 20, characterized in that the alcoholate is a sodium or potassium alcoholate.

32. The method according to claim 26, wherein the removing is a distillation and/or vacuum treatment.
